(19) United States
(12) Patent Application Publication
(10) Pub. No.: US 2002/0048760 A1
(43) Pub. Date: Apr. 25, 2002

(54) Use of mismatch cleavage to detect complementary probes

(75) Inventors: Snezana Drmanac, Palo Alto, CA (US); Chongjun Xu, San Jose, CA (US); Xiao-Hong Qian, Sunnyvale, CA (US)

Correspondence Address: PENNIE & EDMONDS LLP, 1155 Avenue of the Americas, New York, NY, 10036-2711, US

(73) Assignee: Hyseq, Inc.

(21) Appl. No.: 09/825,408

(22) Filed: Apr. 2, 2001

Related U.S. Application Data
(63) Continuation of application No. 09/458,900, filed on Dec. 10, 1999, now abandoned.

(51) Int. Cl. C12Q 1/68

(52) U.S. Cl. 435/6

(57) ABSTRACT

The present invention provides methods and reagents useful for a wide range of nucleic acid sequence analyses, including sequencing by hybridization.

### 1 FIELD OF THE INVENTION

This invention relates in general to methods and reagents for nucleic acid sequence analysis, in particular sequence analysis by hybridization.

### 2 BACKGROUND

The rate of determining the sequence of the four nucleotides in nucleic acid samples is a major technical obstacle for further advancement of molecular biology, medicine, and biotechnology. Nucleic acid sequencing methods which involve separation of nucleic acid molecules in a gel have been in use since 1978. The other proven method for sequencing nucleic acids is sequencing by hybridization (SBH).

The traditional method of determining a sequence of nucleotides (i.e., the order of the A, G, C and T nucleotides in a sample) is performed by preparing a mixture of randomly-terminated, differentially labeled nucleic acid fragments by degradation at specific nucleotides, or by dideoxy chain termination of replicating strands. Resulting nucleic acid fragments in the range of 1 to 500 bp are then separated on a gel to produce a ladder of bands wherein the adjacent samples differ in length by one nucleotide.

The array-based approach of SBH does not require single base resolution in separation, degradation, synthesis or imaging of a nucleic acid molecule. Using mismatch discriminative hybridization of short oligonucleotides k bases in length, lists of constituent k-mer oligonucleotides may be determined for target nucleic acid. Sequence information for the target nucleic acid may be assembled by uniquely overlapping scored oligonucleotides.

There are several approaches available to achieve sequencing by hybridization. In a process called SBH Format 1, nucleic acid samples are arrayed, and labeled probes are hybridized with the samples. Replica membranes with the same sets of sample nucleic acids may be used for parallel scoring of several probes, and/or a multiple label color scheme may be employed (i.e., multiplexing). Multiplexing involves the use of a pool of several probes, each having a different label such as a different fluorescent dye, thereby reducing the number of hybridization cycles and shortening the sequencing process. Nucleic acid samples may be arrayed and hybridized on nylon membranes or other suitable supports. Each membrane array may be reused many times. Format 1 is especially efficient for batch processing large numbers of samples.

In SBH Format 2, probes are arrayed at locations on a substrate which correspond to their respective sequences, and a labeled nucleic acid sample fragment is hybridized to the arrayed probes. In this case, sequence information about a fragment may be determined in a simultaneous hybridization reaction with all of the arrayed probes. For sequencing other nucleic acid fragments, the same oligonucleotide array may be reused. The arrays may be produced by spotting or by in situ synthesis of probes.

In Format 3 SBH, two sets of probes are used. In one embodiment, a set may be in the form of arrays of probes fixed at known positions, and another, labeled set may be stored in multiwell plates. In this case, target nucleic acid need not be labeled. Target nucleic acid and one or more labeled probes are added to the arrayed sets of probes. If one attached probe and one labeled probe both hybridize contiguously on the target nucleic acid, they are covalently ligated, producing a detectable sequence equal to the sum of the length of the ligated probes. The process allows for sequencing long nucleic acid fragments, e.g., a complete bacterial genome, without nucleic acid subcloning in smaller pieces.

The efficiency of all form of sequencing by hybridization depends critically on the ability to distinguish between perfectly matched duplexes and duplexes that contain a single mismatch at any position, referred to as mismatched duplexes or heteroduplexes. To date, however, no fully satisfactory method for achieving the required level of discrimination in all applications has been available. This difficulty arises because the free energy of the interaction between target and probe (generally measured in terms of the Tₘ, or melting temperature, of the duplex formed) varies depending upon the nature of the nucleic acids involved. In particular, the Tₘ for nucleic acid duplexes depends upon the length of the duplex, the base composition of the hybridized nucleic acids (particularly GC content), the propensity of the nucleic acids to form hairpin loops, and the existence of mismatches. The effect of mismatches depends upon the nature of the mismatch, which can be between two naturally-occurring bases (e.g., G:T or G:A) or in some cases involve a non-naturally occurring base (a non-discriminating base such inosine or the universal “M” base described by Nichols et al., Nature 369:492-3 (1994)). The effect also depends upon the location of the mismatch, with an internal mismatch destabilizing the duplex to a greater extent than a mismatch at or near the terminus of the probe.

The Tₘ of a duplex also depends upon hybridization conditions, e.g., pH, ionic strength and the concentration of solution components such as formamide that affect the free energy of the interaction. See e.g., Ausubel et al., Current Protocols in Molecular Biology, Vol. 1-2, John Wiley & Sons (1989); Sambrook et al., Molecular Cloning A Laboratory Manual, 2nd Ed., Vols. 1-3, Cold Springs Harbor Press (1989); and Maniatis et al., Molecular Cloning: A Laboratory Manual, Cold Spring Harbor Laboratory Cold Spring Harbor, N.Y. (1982), all of which are incorporated herein by reference in their entirety. Thus, the stability of the duplex can be altered by varying the temperature and/or the nature of the hybridization solution. The propensity of any given hybridization conditions to stabilize nucleic acid duplexes is commonly referred to as the stringency of the conditions. In general, altering the stringency, for example by varying temperature, will affect the stability of all hybrids to roughly the same extent. In particular, alterations that stabilize perfectly matched duplexes (the goal in most SBH applications) will also stabilize mismatched duplexes. In contrast, alterations that destabilize mismatched duplexes (another important goal in SBH applications) will also destabilize perfectly matched duplexes. As a result of this tension, depending upon the nature of the probes and targets under investigation, in many cases it will be difficult or impossible to provide hybridization conditions that result in the desired stable duplexes involving perfectly matched nucleic acids but that discriminate against all duplexes involving single-base mismatches. For example, the Tₘ of a GC rich duplex with a single mismatch at a probe terminus might be equal or even greater than a perfectly matched AT rich duplex of the same length.

To date, those working in the field have expended a great deal of effort attempting to overcome this dilemma. These methods are often based on the uses of a chemical agent or agents that attenuate the difference in binding energy between AT and GC pairs, thereby reducing the effect of GC content on Tₘ. One such method involves the use of hybridization solvents that contain the quaternary alkylammonium salts tetraethylammonium chloride or tetramethylammonium chloride instead of sodium chloride (Sambrook et al., supra). Other agents reputed to modulate mismatch discrimination include organic solvents such as formamide, glycol, dimethylsulfoxide, dimethylformamide, urea, guanidinium, amino acid analogs such as betaine (Henke et al., Nucl. Acids Res. 19:3957-3958 (1997); Rees et al., Biochemistry 32:137-144 (1993)), polyamines such as spermidine and spermine (Thomas et al., Nucl. Acids Res. 25:2396-2402 (1997)), other positively charged molecules which neutralize the negative charge of the phosphate backbone, detergents such as sodium dodecyl sulfate and sodium lauryl sarcosinate, minor/major groove binding agents, positively charged polypeptides, and intercalating agents such as acridine, ethidium bromide, and anthracine.

While the use of these chemical agents can to some extent attenuate the influence of GC content on Tₘ, the relative Tₘ of perfectly matched duplexes and corresponding single base mismatches will still vary depending upon other parameters, such as probe length, the ability of the probes and/or target to form hairpin loops, and upon the location of the mismatch. As a result, these agents are not amenable to the development of a universally applicable method of differentiating between perfect matches and single-base mismatches, in a manner independent of the length and nature of the probes. To date such a method has not been suggested. As a result, specific conditions must be defined for specific probe sets. However, if a universal method were available, it would greatly enhance the power and efficiency of sequence analysis techniques involving nucleic acid hybridization. Furthermore, is would greatly simplify the determination of suitable hybridization conditions for use in such techniques.

### 3 SUMMARY OF THE INVENTION

The present invention provides methods for discriminating between a perfectly matched polynucleotide duplex and a mismatched polynucleotide duplex. The methods are particularly useful and appropriate in methods of analyzing nucleic acids by hybridization, especially methods of sequencing by hybridization (SBH).

In some embodiments of the invention, the method involves providing a labeled duplex bound to a solid substrate; cleavage of a polynucleotide of the duplex if and only if the duplex is mismatched, thereby releasing the label from the substrate; and then determining whether the label is still associated with the solid substrate. In a preferred embodiment, the label is released from the terminal end of a nucleic acid strand of the duplex that is covalently attached to the solid substrate. In another preferred embodiment, the label is released from the terminal end of a nucleic acid strand of the duplex that is hybridized to a nucleic acid strand that is covalently attached to the solid substrate.

In other embodiments of the invention, the duplex is not bound to a solid substrate, but rather is free in solution. Mismatch discrimination is achieved by specifically cleaving mismatched polynucleotide duplexes and then detecting only those duplexes that are not cleaved, i.e., perfectly matched duplexes. This form of mismatch discrimination is particularly suited for SBH in solution. In a particularly preferred embodiment, non-cleaved, perfectly matched duplexes are detected by mass spectrometry.

The provided methods are particularly suited for uses involving polynucleotide sequence analysis by hybridization. The methods are applicable to de novo sequencing by hybridization, as well as the detection of mutations or single nucleotide polymorphisms in known sequences. The methods are further useful in a variety of clinical applications, including the detection of pathogenic organisms or deleterious genetic mutations. In a preferred aspect of the invention, the method is used in conjunction with Format II or Format III sequencing by hybridization, and in a particularly preferred aspect, the method is used in conjunction with Format III SBH. In another preferred aspect, the method can be used in sequencing by hybridization formats that do not involve chips, e.g., SBH in solution (see, e.g., application Ser. No. 09/277,383, filed Mar. 25, 1999).

In one embodiment of the invention, the polynucleotide is cleaved by a mismatch specific endonuclease, e.g., T4 endonuclease VII or CelI endonuclease, or a cocktail made up of a plurality of mismatch specific nucleases. In another embodiment, the polynucleotide is cleaved by a chemical reagent.

In some embodiments of the invention, the detectable label is a fluorophore, radioisotope, enzyme, dye, electrophore mass label (EML), or a ligand capable of detection.

### 4 BRIEF DESCRIPTION OF THE FIGURES

The detailed description of the invention may be better understood in conjunction with the accompanying figures as follows:

FIG. 1 is a listing of oligonucleotide probes attached to a glass slide for use in Format III SBH, as described in the Example.

FIG. 2 shows the results of an experiment to determine the extent of mismatch discrimination in Format III SBH that is achieved by treatment with T4 endonuclease VII after the hybridization/ligation reaction, as described in the Example.

FIG. 3 shows the results of experiments to determine the extent of mismatch discrimination in Format III SBH that is achieved by treatment with T4 endonuclease VII during a 1 hour (FIG. 3A) and 4 hour (FIG. 3B) hybridization/ligation reaction, as described in the Example.

FIG. 4 provides a comparison of the degree of mismatch discrimination achieved in Format III SBH by a one hour versus a four hour hybridization/ligation in the presence of T4 endonuclease VII, as described in the Example.

### 5 DETAILED DESCRIPTION OF THE INVENTION

5.1 Introduction

The present invention affords a solution to the problem of mismatch hybridization identified supra, by providing an improved method for discrinating between perfectly matched duplexes and duplexes that contain one or more mismatches at any position, particularly in the case where the duplexes are detectably labeled and bound to a solid support. The invention employs mismatch-dependent cleavage to discriminate between perfectly matched and mismatched duplexes. Mismatch-dependent cleavage refers to a mode of polynucleotide cleavage that is specific for polynucleotides that are involved in a mismatched duplex, and does not result in the cleavage of polynucleotides involved in a perfectly matched duplex. As described below, in one embodiment of the invention mismatch-dependent cleavage can be used to selectively detach a detectable label from mismatched duplexes, leaving perfectly matched duplexes labeled. Thus, detection of a the label will only identify sequences involved in perfectly matched duplexes. In another embodiment, mismatch-dependent cleavage can be used to selectively detach a signal quenching group from mismatched duplexes, in this case resulting in an enhanced signal. Furthermore, mismatch-dependent cleavage can be used to discriminate between perfectly matched duplexes in a variety of SBH applications, including applications that involve hybridization to an immobilized probe and applications that involve hybridization in solution. Mismatch-dependent cleavage reduces the need for determining and using precise hybridization stringency conditions to discriminate perfect matches from mismatches and improves the accuracy and usefulness of many application requiring hybridization, especially SBH.

SBH is a well developed technology that may be practiced by a number of methods known to those skilled in the art. Specifically, the techniques related to sequencing by hybridization discussed in the following documents are incorporated by reference herein: Drmanac et al., U.S. Pat. No. 5,202,231; Drmanac et al., Genomics 4:114-128 (1989); Drmanac et al., Proceedings of the First Int'l. Conf. Electrophoresis Supercomputing Human Genome, Cantor et al. eds, World Scientific Pub. Co., Singapore, 47-59 (1991); Drmanac et al., Science 260:1649-1652 (1993); Lehrach et al., Genome Analysis: Genetic and Physical Mapping 1:39-81 (1990), Cold Spring Harbor Laboratory Press; Drmanac et al., Nucl. Acids Res. 14(11):4691-2 (1986); Drmanac et al., J. Biomol. Struct. Dyn. 8(5):1085-102 (1991); Hoheisel et al., Mol. Gen. 220(4):903-14:125-132 (1991); Strezoska et al., Proc. Nat'l. Acad. Sci. (USA) 88(22):10089-93 (1991); Drmanac et al., Nucl. Acids Res. 19:5839-42 (1991); Drmanac et al., Nature Biotechnology 16:54-58 (1998); Sanger et al., Proc. Nat'l. Acad. Sci. (USA) 74: 5463-5467 (1977); Maxam & Gilbert, Proc. Nat'l. Acad. Sci. (USA) 74: 560-564 (1977); Holey et al., Science 147:1462-1465 (1965); Hunkapillar et al., Science 254:59-63 (1991); Doty et al., Proc. Nat 'l. Acad. Sci. (USA) 46: 461-466 (1990); Beaucage & Caruthers, Tetrahedron Lett. 22:1859-1862 (1981); Wallace et al., Nucl. Acids Res. 6:3543-3557 (1979); Saiki et al., Proc. Nat'l. Acad. Sci. (USA) 86:6230-6234 (1989); Poustka & Lehrach, Trends Genet. 2:174-179 (1986); Bains & Smith, J. Theor. Biol. 135:303-307 (1988); Southern, WO 89/10977; Southern, U.S. Pat. No. 5,700,637; Lysov et al., Dokl. Akad. Nauk. SSSR 303:1508-1511 (1988); Pevzner & Lipschutz, “Towards DNA Sequencing Chips,” in: Mathematical Foundations of Computer Science (1994); Privara et al., Eds., pp. 143-158, The Proceedings of the 19^{th} International Symposium, MFCS '94, Kosice, Slovakia, Springer-Verlag, Berlin (1995); Khrapko, FEBS Lett. 256:118-122 (1989); Dramanac & Crkvenjakov, Int'l. J. Genome Res. 1(1):59-79 (1992); Broude et al., Proc. Nat'l. Acad. Sci. (USA) 91:3072-3076 (1994); Drmanac, WO 95/09248; Nikiforov et al., Nucl. Acids Res. 22:4167-4175 (1994); Hoheisel et al., Cell 73:109-120 (1993); Labat & Drmanac, “Simulations of Ordering and sequence Resconstruction of Random DNA Clones Hybridized with a Small Number of Oligomer Probes,” in: The Second International Conference on Electrophoresis, Supercomputing and the Human Genome, pp. 555-565, World Scientific, Singapore, Malaysia (1992); Scholler et al., Nucl. Acids Res. 23:3842-3849 (1995); Drmanac et al., “Partial Sequencing by Hybridization: Concept and Applications in Genome Analysis,” in: The First International Conference on Electrophoresis, Supercomputing and the Human Genome, pp. 60-74, World Scientific, Singapore, Malaysia (1991); Drmanac et al., Genomics 37:2940 (1996); Milosavljevic et al., Genome Res. 6:132-141 (1996); Meier et al., Nucl. Acids Res., 26:2216-2223 (1998); Milosavljevic et al., Genomics 37:77-86 (1996); Lockhart, et al., Nat. Biotechnol. 14:1675-1680 (1996); Cheng et al., Nat. Biotechnol. 16:541-546 (1998); Wang et al., Science 280:1077-1082 (1998); Dramanc & Crkvenjakov, Scientia Yugoslaviea 16:99-107 (1990); Strezoska et al., Proc. Nat'l. Acad. Sci. (USA) 88:10089-10093 (1991); Drmanac et al., Nat. Biotech. 16:54-58 (1998); Southern et al., Genomics 13:1008-1017 (1992); Chee et al., Science 274:610-614 (1996); Kozal et al., Nature Medicine 7:753-759 (1996); Hacia et al., Nature Genetics 14:441-447 (1996); Hacia et al., Genome Res. 8:1245-1258(1998); Gunderson et al., Genome Res. 8:1142-1153 (1998); Drmanac et al., Science 260:1649-1652 (1993); Drmanac et al., Electrophoresis 13:566-573 (1992); Drmanac & Drmanac, Meth. Enzymology 303:165-178 (1999); Wetmur, Crit. Rev. Biochem, Mol. Biol. 26:227-259 (1991); Breslauer et al., Proc. Nat'l. Acad. Sci. (USA) 83:3746-3750 (1986); Sugimoto et al., Nucl. Acid Res. 24:4501-4505 (1996); Michiels et al., CABIOS 3:203-210 (1987); Drmanac et al., DNA and Cell Biol. 9(7):527-534 (1994); Housby & Southern, Nucl. Acids Res. 26:4259-4266 (1998); Dianzani et al., Genomics 11:48-53 (1991).

5.2 Preparation and Labeling of Polynucleotides

The practice of the instant invention employs a variety of polynucleotides. Typically some of the polynucleotides are detectably labeled. Species of polynucleotides used in the practice of the invention include target nucleic acids and probes.

The term “probe” refers to a relatively short polynucleotide, preferably DNA. Probes are preferably shorter than the target nucleic acid by at least one base, and more preferably they are 25 bases or fewer in length, still more preferably 20 bases or fewer in length. Of course, the optimal length of a probe will depend on the length of the target nucleic acid being analyzed. For a target nucleic composed of about 100 or fewer bases, the probes are preferably at least 7-mers; for a target of about 100-200 bases, the probes are preferably at least 8-mers; for a target nucleic acid of about 200400 bases, the probes are preferably at least 9-mers; for a target nucleic acid of about 400-800 bases, the probes are preferably at least 10-mers; for a target nucleic acid of about 800-1600 bases, the probes are preferably at least 11-mers; for a target of about 1600-3200 bases, the probes are preferably at least 12-mers, for a target of about 3200-6400 bases, the probes are preferably at least 13-mers; and for a target of about 6400-12,800 bases, the probes are preferably at least 14-mers. For every additional two-fold increase in the length of the target nucleic acid, the optimal probe length is one additional base.

Those of skill in the art will recognize that for format III SBH applications, the above-delineated probe lengths are post-ligation. Thus, as used throughout, specific probe lengths refer to the actual length of the probes for format II-type SBH applications and the lengths of ligated probes in format III-type SBH. Probes are normally single stranded, although double-stranded probes may be used in some applications.

While typically the probes will be composed of naturally-occurring bases and native phosphodiester backbones, they need not be. For example, the probes may be composed of one or more modified bases, such as 7-deazaguanosine or the universal “M” base, or one or more modified backbone interlinkages, such as a phosphorothioate. The only requirement is that the probes be able to hybridize to the target nucleic acid. A wide variety of modified bases and backbone interlinkages that can be used in conjunction with the present invention are known, and will be apparent to those of skill in the art.

The length of the probes described above refers to the length of the informational content of the probes, not necessarily the actual physical length of the probes. The probes used in SBH frequently contain degenerate ends that do not contribute to the information content of the probes. For example, SBH applications frequently use mixtures of probes of the formula NₓB_{y}N_{z}, wherein N represents any of the four bases and varies for the polynucleotides in a given mixture, B represents any of the four bases but is the same for each of the polynucleotides in a given mixture, and x, y, and z are integers. Typically, x and z are independently integers between 0 and 5 and y is an integer between 4 and 20. The number of known bases B_{y} defines the “information content” of the polynucleotide, since the degenerate ends do not contribute to the information content of the probes. Linear arrays comprising such mixtures of immobilized polynucleotides are useful in, for example, sequencing by hybridization. Hybridization discrimination of mismatches in these degenerate probe mixtures refers only to the length of the informational content, not the full physical length.

Probes for use in the instant invention may be prepared by techniques well known in the art, for example by automated synthesis using an Applied Biosystems synthesizer. Alternatively, probes may be prepared using Genosys Biotechnologies Inc. methods using stacks of porous Teflon wafers. For purposes of this invention, the source of oligonucleotide probes used is not critical, and one skilled in the art will recognize that oligonucleotides prepared using other methods currently known or later developed will also suffice.

The term “target nucleic acid” refers to a polynucleotide, or some portion of a polynucleotide, for which sequence information is desired, typically the polynucleotide that is sequenced in the SBH assay. The target nucleic acid can be any number of nucleotides in length, depending on the length of the probes, but is typically on the order of 100, 200, 400, 800, 1600, 3200, 6400, or even more nucleotides in length. The target nucleic acid may be composed of ribonucleotides, deoxyribonucleotides or mixtures thereof. Typically, the target nucleic acid is a DNA. While the target nucleic acid can be double-stranded, it is preferably single stranded. Moreover, the target nucleic acid can be obtained from virtually any source. Depending on its length, it is preferably sheared into fragments of the above-delineated sizes prior to use an SBH assay. Like the probes, the target nucleic acid can be composed of one or more modified bases or backbone interlinkages.

The target nucleic acid may be obtained from any appropriate source, such as cDNAs, genomic DNA, chromosomal DNA, microdissected chromosome bands, cosmid or YAC inserts, and RNA, including mRNA without any amplification steps. For example, Sambrook et al. (1989) describes three protocols for the isolation of high molecular weight DNA from mammalian cells (p. 9.14-9.23).

The polynucleotides would then typically be fragmented by any of the methods known to those of skill in the art including, for example, using restriction enzymes as described at 9.24-9.28 of Sambrook et al. (1989), shearing by ultrasound, and NaOH treatment. A particularly suitable method for fragmenting DNA utilizes the two base recognition endonuclease, CviJI, described by Fitzgerald et al., Nucleic Acids Res., 20(14):3753-62 (1992).

In a preferred embodiment, the target nucleic acids are prepared so that they cannot be ligated to each other, for example by treating the fragmented nucleic acids obtained by enzyme digestion or physical shearing with a phosphatase (e.g., calf intestinal phosphatase). Alternatively, nonligatable fragments of the sample nucleic acid may be obtained by using random primers (e.g., N₅-N₉, where N=A, G, T, or C), which have no phosphate at their 5′-ends, in a Sanger-dideoxy sequencing reaction with the sample nucleic acid.

In most cases it is important to denature the DNA to yield single stranded pieces available for hybridization. This may be achieved by incubating the DNA solution for 2-5 minutes at 80-90° C. The solution is then cooled quickly to 2° C. to prevent renaturation of the DNA fragments before they are contacted with the probes.

Depending on the format used, probes and/or target nucleic acids may be detectably labeled. Virtually any label that produces a detectable signal and that is capable of being immobilized on a substrate or attached to a polynucleotide can be used in conjunction with the arrays of the invention. Preferably, the signal produced is amenable to quantification. Suitable labels include, by way of example and not limitation, radioisotopes, fluorophores, chromophores, chemiluminescent moieties, etc.

Due to their ease of detection, polynucleotides labeled with fluorophores are preferred. Fluorophores suitable for labeling polynucleotides are described, for example, in the Molecular Probes catalog (Molecular Probes, Inc., Eugene Oreg. 97402-9144), and the references cited therein. Methods for attaching fluorophore labels to polynucleotides are well known, and can be found, for example in Goodchild, Bioconjug Chem. 1(3):165-87 (1990). A preferred fluorophore label is the carboxylic acid of tetramethyl rhodaimine (TAMRA dye), which is available from Molecular Probes.

In another preferred embodiment, the different labels are electrophore mass labels (“EML”), which can be detected by electron capture mass spectrometry (EC-MS). EMLs may be prepared from a variety of backbone molecules, with certain aromatic backbones being particularly preferred, e.g., see Xu et al., J. Chromatog. 764:95-102 (1997). The EML is attached to a probe in a reversible and stable manner, and after the probe is hybridized to target nucleic acid, the EML is removed from the probe and identified by standard EC-MS (e.g., the EC-MS may be done by a gas chromatograph-mass spectrometer).

Polynucleotides can also be labeled with enzymes capable of catalyzing the production of a detectable product. For example, Cate et al., Genet. Anal. Tech. Appl., 8(3):102-6 (1991), describe the use of oligonucleotide probes directly conjugated to alkaline phosphatase in combination with a direct chemiluminescent substrate (AMPPD) to allow probe detection. Horse radish peroxidase is another example of a suitable enzyme.

Polynucleotides can also be labeled with a ligand capable of detection via association with a detectable entity capable of binding to the ligand. Biotin is an example of a ligand capable of detection, and can be detected via its association with an avidin or streptavidin containing compound, e.g., streptavidin linked to an enzyme capable of generating a detectable product, such as alkaline phosphatase.

Alternatively, the probes or targets may be labeled by any other technique known in the art. Preferred techniques include direct chemical labeling methods and enzymatic labeling methods, such as kinasing and nick-translation. Labeled probes could readily be purchased form a variety of commercial sources, including GENSET, rather then synthesized.

In general, the label can be attached to any part of the probe or target polynucleotide, including the free terminus or one or more of the bases. In preferred embodiments, the label is attached to a terminus of the polynucleotide. The label, when attached to a solid support by means of a polynucleotide, must be located such that it can be released for the solid support by cleavage with a mismatch specific endonuclease, as described infra. Preferably, the position of the label will not interfere with hybridization, ligation, cleavage or other post-hybridization modifications of the labeled polynucleotide.

Some embodiments of the invention employ multiplexing, i.e., the use of a plurality of distinguishable labels (such as different fluorophores, chromophores, or radioactive labels, or mixtures thereof). Multiplexing allows the simultaneous detection of a plurality of sequences in one hybridization reaction. For example, a multiplex of 4 colors reduces the number of hybridizations required by an additional factor of 4.

Other embodiments employ the use of informative pools of probes to reduce the redundancy normally found in SBH protocols, thereby reducing the number of hybridization reactions needed to unambiguously determine a target DNA sequence. Informative pools of probes and methods of using the same can be found in U.S. Provisional Patent Application Ser. No. 60/115,284, which is incorporated herein by reference in its entirety.

5.3 Attachment of Polynucleotides to a Solid Substrate

Some embodiment of the instant invention requires polynucleotides, typically referred to as immobilized probes, to be attached to a solid substrate. In preferred embodiments, the appropriate probes or pools of probes are attached to a solid substrate in a defined, spatially-addressable manner so as to provide spatially-addressable polynucleotide arrays. The composition of the immobilized polynucleotides is not critical. The only requirement is that they be capable of hybridizing to a target nucleic acid of complementary sequence.

Spatially-addressable polynucleotide arrays come in many forms, and can be either one-dimensional, two-dimensional or three-dimensional. Arrays may be designed for various applications (e.g. mapping, partial sequencing, sequencing of targeted regions for diagnostic purposes, mRNA sequencing and large scale sequencing). A two-dimensional array will typically consist of an appropriate collection of polynucleotides attached to the surface of a flat, planar substrate, such as a glass slide, to form a two-dimensional grid or matrix. The arrays are spatially-addressable in the sense that the polynucleotides composing the array are known, and are therefore defined by their spatial addresses (xy coordinates).

One-dimensional, i.e., linear, spatially-addressable arrays can be generated by the attachment of polynucleotides to beads, a different polynucleotide sequence corresponding to each bead, and arranging the beads in a known, single-file order, e.g., in a capillary tube or etched channel. Spatially-addressable linear arrays are described U.S. patent application Ser. Nos. 09/251,305 and 09/083,861, which are incorporated herein by reference in their entirety.

Three-dimensional arrays are comprised of multiple layers, and each layer may be analyzed separate and apart from the other layers. Spatially-addressable three dimensional array may take a number of forms, for example, the array may be disposed on a substrate having multiple depressions with probes located at different depths within the depressions (each level is made up of probes at similar depths within the depression); or the array may be disposed on a substrate having depressions of different depths with the probes located at the bottom of the depression, or at the peaks separating the depressions or some combination of peaks and depressions may be used (each level is made up of all the probes at a certain depth); or the array may be disposed on a substrate comprised of multiple sheets that are layered to form a three-dimensional array. Alternatively, a three-dimensional array can be made by stacking linear arrays.

Multiple arrays can also be arranged in a sandwich configuration. This arrangement allows multiple probes to be assayed simultaneously with one probe mixture. Sandwich arrays and their methods of use are described in U.S. patent application Ser. Nos. 09/251,303 and 09/085,529, which are incorporated herein by reference in their entirety.

The nature and geometry of the solid substrate will depend upon a variety of factors, including, among others, the type of array (e.g., one-dimensional, two-dimensional or three-dimensional) and the mode of attachment (e.g., covalent or non-covalent). Generally, the substrate can be composed of any material which will permit immobilization of the polynucleotide and which will not melt or otherwise substantially degrade under the conditions used to hybridize and/or denature nucleic acids. In addition, where covalent immobilization is contemplated, the substrate should be activatable with reactive groups capable of forming a covalent bond with the polynucleotide to be immobilized.

A number of materials suitable for use as substrates in the instant invention have been described in the art. Exemplary suitable materials include, for example, acrylic, styrene-methyl methacrylate copolymers, ethylene/acrylic acid, acrylonitrile-butadiene-styrene (ABS), ABS/polycarbonate, ABS/polysulfone, ABS/polyvinyl chloride, ethylene propylene, ethylene vinyl acetate (EVA), nitrocellulose, nylons (including nylon 6, nylon 6/6, nylon 6/6-6, nylon 6/9, nylon 6/10, nylon 6/12, nylon 11 and nylon 12), polycarylonitrile (PAN), polyacrylate, polycarbonate, polybutylene terephthalate (PBT), polyethylene terephthalate (PET), polyethylene (including low density, linear low density, high density, cross-linked and ultra-high molecular weight grades), polypropylene homopolymer, polypropylene copolymers, polystyrene (including general purpose and high impact grades), polytetrafluoroethylene (PTFE), fluorinated ethylene-propylene (FEP), ethylene-tetrafluoroethylene (ETFE), perfluoroalkoxyethylene (PFA), polyvinyl fluoride (PVF), polyvinylidene fluoride (PVDF), polychlorotrifluoroethylene (PCTFE), polyethylene-chlorotrifluoroethylene (ECTFE), polyvinyl alcohol (PVA), silicon styrene-acrylonitrile (SAN), styrene maleic anhydride (SMA), metal oxides and glass.

The substrate may be in the form of beads, particles or sheets, and may be permeable or impermeable, depending on the type of array. For example, for linear or three-dimensional arrays the substrate may consist of bead or particles (such as conventional solid phase synthesis supports), fibers (such as glass wool or other glass or plastic fibers) or glass or plastic capillary tubes. For two-dimensional arrays, the substrate is preferably in the form of plastic or glass sheets in which at least one surface is substantially flat. Particularly preferred substrates for use with two-dimensional arrays are glass slides.

A variety of techniques have been described for synthesizing and/or immobilizing spatially-addressable arrays of polynucleotides, including in situ synthesis, where the polynucleotides are synthesized directly on the surface of the substrate (see, e.g., U.S. Pat. No. 5,744,305) and attachment of pre-synthesized polynucleotides to the surface of a substrate at discrete locations (see, e.g., WO 98/31836). Additional methods are described in WO 98/31836 at pages 41-45 and 47-48, among other places. The present invention is suitable for use with any of these currently available, or later developed, techniques.

In general, oligonucleotides may be bound to a support through appropriate reactive groups. Such groups are well known in the art and include, for example, amino (—NH₂); hydroxyl (—OH); or carboxyl (CO₂H) groups. Support bound oligonucleotides may be prepared by any of the methods known to those of skill in the art using any suitable support such as glass, polystyrene or Teflon. One strategy is to precisely spot oligonucleotides synthesized by standard synthesizers. Immobilization can be achieved by many methods, including, for example, using passive adsorption (Inouye & Hondo, J. Clin. Microbiol. 28(6): 1469-72 (1990)); using UV light (Dahlen et al., Mol. Cell Probes 1(2): 159-68 (1987)); or by covalent binding of base modified DNA (Keller et al., Anal. Biochem. 170(2):441-50 (1988); (Keller et al., Anal. Biochem., 177(2):392-5 (1989)); or by formation of amide groups between the probe and the support (Zhang et al., Nucleic Acids Res. 19(14):3929-33 (1991)); all references being specifically incorporated herein.

Another strategy that may be employed is the use of the strong biotin-streptavidin interaction as a linker. For example, Broude et al., Proc. Natl. Acad. Sci. USA, 91(8):3072-6 (1994), describe the use of biotinylated probes, although these are duplex probes, that are immobilized on streptavidin-coated magnetic beads. Streptavidin-coated beads may be purchased from Dynal, Oslo. Of course, this same linking chemistry is applicable to coating any surface with streptavidin. Biotinylated probes may be purchased from various sources, such as, e.g., Operon Technologies (Alameda, Calif.).

Nunc Laboratories (Naperville, Ill.) is also selling suitable material that could be used. Nunc Laboratories have developed a method by which DNA can be covalently bound to the microwell surface termed Covalink NH. CovaLink NH is a polystyrene surface grafted with secondary amino groups that serve as bridge-heads for further covalent coupling. CovaLink Modules may be purchased from Nunc Laboratories. DNA molecules may be bound to CovaLink exclusively at the 5′-end by a phosphoramidate bond, allowing immobilization of more than 1 pmol of DNA (Rasmussen et al., Anal. Biochem. 198(1):13842 (1991)).

The use of CovaLink NH strips for covalent binding of DNA molecules at the 5′-end has been described (Rasmussen et al., 1991). In this technology, a phosphoramidate bond is employed. This is beneficial as immobilization using only a single covalent bond is preferred. The phosphoramidate bond joins the DNA to the CovaLink NH secondary amino groups that are positioned at the end of spacer arms covalently grafted onto the polystyrene surface through a 2 nm long spacer arm.

It is contemplated that a further suitable method for use with the present invention is that described in PCT Patent Application WO 90/03382, incorporated herein by reference. This method of preparing an oligonucleotide bound to a support involves attaching a nucleoside 3′-reagent through the phosphate group by a covalent phosphodiester link to aliphatic hydroxyl groups carried by the support. The oligonucleotide is then synthesized on the supported nucleoside and protecting groups removed from the synthetic oligonucleotide chain under standard conditions that do not cleave the oligonucleotide from the support. Suitable reagents include nucleoside phosphoramidite and nucleoside hydrogen phosphorate.

Alternatively, addressable laser-activated photodeprotection may be employed in the chemical synthesis of oligonucleotides directly on a glass surface, as described by Fodor et al., Science 251:767-73 (1991), incorporated herein by reference.

One particular way to prepare support bound oligonucleotides is to utilize the light-generated synthesis described by Pease et al. (1994) Proc. Natl. Acad. Sci. (USA) 91(11):5022-6, incorporated herein by reference. These authors used current photolithographic techniques to generate arrays of immobilized oligonucleotide probes, i.e., DNA chips. These methods, in which light is used to direct the synthesis of oligonucleotide probes in high-density, miniaturized arrays, utilize photolabile 5′-protected N-acyl-deoxynucleoside phosphoramidites, surface linker chemistry and versatile combinatorial synthesis strategies. A matrix of 256 spatially defined oligonucleotide probes may be generated in this manner and then used in the advantageous Format III sequencing, as described herein.

Although the in situ synthesis methods described utilize phosphoramidite reagents, it will be recognized that other reagents utilizing other synthesis strategies can also be employed, and in certain circumstances may be preferable, depending on the stability of the chosen label to the synthesis conditions. Non-limiting examples of suitable chemistries and reagents are described, for example in Oligonucleotide Synthesis: A Practical Approach, M. J. Gait, Ed., IRL Press, Oxford, England, 1985.

Alternatively, one could purchase a spatially-addressable polynucleotide array, such as one of the light-activated chips described above, from a commercial source. In this regard, one may contact Affymetrix of Santa Clara, Calif., and Beckman.

In a preferred embodiment, the probes of the invention are connected to the solid substrate by means of a linker moiety. The linker may be comprised of atoms capable of forming at least two covalent bonds such as carbon, silicon, oxygen, sulfur, phosphorous, and the like, or may be comprised of molecules capable of forming at least two covalent bonds such as sugar-phosphate groups, amino acids, peptides, nucleosides, nucleotides, sugars, carbohydrates, aromatic rings, hydrocarbon rings, linear and branched hydrocarbons, and the like. In a particularly preferred embodiment of the invention the linker moiety is composed of alkylene glycol moieties.

In some preferred embodiments, a label is attached to the solid substrate along with the probe or pool of probes. The label can be separate or attached to the probe or probes of interest, so long as the extent of label attachment is proportional to the amount of probe attached. The intensity of the signal from the attached label at a given location on the substrate provides an assessment of the amount of probe attached at that location, and thus provides a way to verify the quality and integrity of the array as a whole. The intensity information is also useful when the array is interrogated, as it provides a way to distinguish real differences in signal intensity due to experimental results from artifactual variations due simply to inconsistent attachment of probe to substrate. Such array verification methods are described in provisional U.S. patent application Ser. No. 60/111,961, which is incorporated herein by reference in its entirety.

5.4 Formation of Detectably Labeled Duplexes on a Solid Support

In a preferred aspect of the invention, a detectable label, as part of a labeled polynucleotide, is specifically attached to a spatially-addressable polynucleotide array. In one preferred embodiment of the invention, a labeled target nucleic acid is bound by means of complementary base-pairing interactions to a probe that is itself attached to a solid substrate as part of a spatially-addressable polynucleotide array, thereby forming a duplex. In another preferred embodiment, a labeled probe is covalently attached, i.e., ligated, to another probe that is attached to solid substrate as part of a spatially-addressable polynucleotide array, if the two probes hybridize to a target nucleic acid in a contiguous fashion.

As used herein, nucleotide bases “match” or are “complementary” if they form a stable duplex or binding pair under specified conditions. The specificity of one base for another is dictated by the availability and orientation of hydrogen bond donors and acceptors on the bases. For example, under conditions commonly employed in hybridization assays, adenine (“A”) matches thymine (“T”), but not guanine (“G”) or cytosine (“C”). Similarly, G matches C, but not A or T. Other bases which interact in less specific fashion, such as inosine or the Universal Base (“M” base, Nichols et al., 1994), or other modified bases, for example methylated bases, are complementary to those bases for which they form a stable duplex under specified conditions. Nucleotide bases which are not complementary to one another are termed “mismatches.”

A pair of polynucleotides, e.g., a probe and a target nucleic acid, are termed “complementary” or a “match” if, under specified conditions, the nucleic acids hybridize to one another in an interaction mediated by the pairing of complementary nucleotide bases, thereby forming a duplex. A duplex formed between two polynucleotides may include one or more bases mismatches. Such a duplex is termed a “mismatched duplex,” or heteroduplex. The less stringent the hybridization conditions are, the more likely it is that mismatches will be tolerated and relatively stable mismatched duplexes can be formed. In a preferred embodiment of the invention, all mismatches in a mismatched duplex include one of the five naturally-occurring nucleotide bases, i.e., adenine, guanine, thymine, cytosine, or uracil. In particularly preferred embodiments, both of the bases involved any mismatch are members of the family of five naturally-occurring bases.

A subset of matched polynucleotides, termed “perfectly complementary” or “perfectly matched” polynucleotides, is composed of pairs of polynucleotides containing continuous sequences of bases that are complementary to one another and wherein there are no mismatches (i.e., absent any surrounding sequence effects, the duplex formed has the maximal binding energy for the particular nucleic acid sequences). “Perfectly complementary” and “perfect match” are also meant to encompass polynucleotides and duplexes which have analogs or modified nucleotides. A “perfect match” for an analog or modified nucleotide is judged according to a “perfect match rule” selected for that analog or modified nucleotide (e.g., the binding pair that has maximal binding energy for a particular analog or modified nucleotide).

In the case where a pool of probes with degenerate ends of the type NₓB_{y}N_{z}, is used, as described above, a perfect match encompasses any duplex where the information content regions, i.e., the B_{y} regions, of the probes are perfectly matched. Discrimination against mismatches in the N regions will not affect the results of a hybridization experiment, since such mismatches do not interfere with the information derived from the experiment.

In a preferred embodiment of the invention, detectable label is attached to a spatially-addressable polynucleotide array in the context of Format II SBH. In this case, a detectably labeled target nucleic acid is hybridized to an array of probes. In the context of this invention, it is not critical that the hybridization conditions be stringent, since mismatched complexes will not be detected as a result of the selective removal of label in the next step. The hybridization conditions must permit the formation of perfectly matched hybrids between the probes and labeled target nucleic acid. Guidelines for conducting hybridization can be found in papers such as Drmanac et al., DNA Cell Biol. 9:527-34(1990); Khrapko et al., DNA Seq. 1:375-88 (1991); and Broude et al., Proc. Natl. Acad. Sci. (USA) 91:3072-76 (1994), which are incorporated herein by reference in their entirety. Hybridization solutions used can, but need not, include chemical agents that attenuate the difference in binding energy between AT and GC pairs. Preferably, target nucleic acids used in Format II SBH are labeled only at a terminus.

In a particularly preferred embodiment of the invention, detectable label is attached to a spatially-addressable polynucleotide array in the context of Format III SBH. In Format III, a set of immobilized oligonucleotide probes of known sequence is provided on a solid substrate under conditions which permit them to hybridize with target nucleic acids having respectively complementary sequences. A second set of labeled oligonucleotide probes is provided in solution. Both within the sets and between the sets the probes may be of the same length or of different lengths. A target nucleic acid to be sequenced, or intermediate fragments thereof, may be applied to the first set of probes in double-stranded form (especially where a recA protein is present to permit hybridization under non-denaturing conditions), or in single-stranded form and under conditions which permit hybrids of different degrees of complementarity. Guidelines for determining appropriate hybridization conditions can be found in papers such as Drmanac et al. (1990), Khrapko et al. (1991), Broude et al. (1994) (all cited supra) and WO 98/31836, which is incorporated herein by reference in its entirety. These articles teach the ranges of hybridization temperatures, buffers and washing steps that are appropriate for use in the initial steps of Format III SBH. The target nucleic acid is applied to the first set of probes before, after or simultaneously with the second set of probes.

Probes that hybridize to contiguous sites on the target are covalently attached to one another, or ligated. Ligation may be implemented by a chemical ligating agent (e.g. water-soluble carbodiimide or cyanogen bromide); by a ligase enzyme, such as the commercially available T₄ DNA ligase; by stacking interactions; or by any other means of causing chemical bond formation between the adjacent probes.

For example, appropriate conditions for chemical ligation using a carbodiimide are as follows: 50 mM 2-[N-morpholino]ethanesulfonic acid (MES) (pH 6.0 with KOH), 10 mM MgCl₂, 0.001% SDS, 200 mM 1-ethyl-3-(3-dimethylaminopropyl)carbodiimide hydrochloride (EDC), 50 mM imidazole (pH 6.0 with HCl) for 14 hr at 30° C. Optionally, 3-4 M tetramethylamonium chloride (TMACl) can be included in the ligation to help normalize the intensities of A/T-rich and G/C-rich probes. However, the use of TMACl is not critical owing to the mismatch discrimination achieved by the mismatch-dependent cleavage step of the disclosed methods.

In preferred embodiments, a DNA ligase, e.g., T4 DNA ligase, is used. Appropriate conditions for enzymatic ligation using T4 DNA ligase are as follows: 50 mM Tris (pH 7.8), 10 mM MgCl₂, 1 mM ATP, 50 μg/ml bovine serum albumin (BSA), 10% PEG, 3 unit/μl T4 DNA ligase for 30 minutes at room temperature.

After permitting adjacent probes to be ligated, target nucleic acids and labeled probes which are not bound to the substrate by chemical bonding to a member of the immobilized set of probe are washed away, for example, using a high temperature (up to 100° C.) wash solution which melts hybrids. Normally, the labeled probes will only be labeled on the terminus not capable of being ligated to a free end of one of the first set of immobilized probes. As a result, after attachment, label is present only at the free terminus of ligated, immobilized probes.

The hybridization is typically carried out for up to several hours in high salt concentrations at a low temperature (−2° C. to 5° C.) because of a relatively low concentration of target DNA that can be provided. Optionally, chemical agents that enhance discrimination between perfectly complementary duplexes and single-base mismatches can be used. In some preferred embodiments a washing step is performed prior to the addition of labeled probe. Preferably, the same buffer is used for the hybridization and washing steps.

After washing, the present invention particularly contemplates adding labeled probes and incubating for several minutes only (because of the high concentration of added oligonucleotides) at a low temperature (0-5° C.), then increasing the temperature to 3-10° C., depending on the length of the labeled and immobilized probes, followed by the addition of washing buffer. The washing buffer should be compatible with the subsequent ligation reaction (e.g., 100 mM salt concentration range). After adding ligase, the temperature is increased again to 15-37° C., which speeds ligation (to less than 30 min).

In some embodiments of the invention, cationic detergents can be included in hybridization buffers for Format III SBH, as described by Pontius & Berg, Proc. Natl. Acad. Sci. (USA) 88(18):823741 (1991), incorporated herein by reference. These authors describe the use of two simple cationic detergents, dodecy- and cetyltrimethylammonium bromide (DTAB and CTAB) in DNA renaturation.

When using a ligase enzyme, it can be added with the labeled probes or after the proper washing step to reduce the background. The use of DNA ligases is well established within the field of molecular biology. For example, Hood and colleagues described a ligase-mediated gene detection technique (Landegren et al., Science, 241:1077-80 (1988)), the methodology of which can be readily adapted for use in Format III SBH. Wu & Wallace, Gene, 76(2):245-54 (1989), also describe the use of bacteriophage T4 DNA ligase to join two adjacent, short synthetic oligonucleotides. Their oligo ligation reactions were carried out in 50 mM Tris HCl pH 7.6, 10 mM MgCl₂, 1 mM ATP, 1 mM DTT, and 5% PEG. Ligation reactions were heated to 100° C. for 5-10 min followed by cooling to 0° C. prior to the addition of T4 DNA ligase (1 unit; Bethesda Research Laboratory). Most ligation reactions were carried out at 30° C. and terminated by heating to 100° C. for 5 min.

In some embodiments, a post-ligation washing appropriate for discriminating detection of hybridized adjacent, or ligated, oligonucleotides is then performed to remove non-ligated labeled probes. If this post-ligation washing is performed, it is important that target nucleic acid does not dissociate from ligated probes, but remains present for the mismatch-specific endonuclease step described infra.

5.5 Mismatch-specific Release of Detectable Label from Solid Support

A critical element of the instant invention is the release of detectable label from nucleic acids involved in mismatched duplexes by mismatch-dependent cleavage, preferably by means of a mismatch-specific cleavage agent, more preferably a mismatch-specific nuclease or nuclease cocktail. The term “mismatch-dependent cleavage” refers to the preferential cleavage of mismatched polynucleotides, i.e., polynucleotides involved in a mismatched duplex, relative to perfectly matched polynucleotides. In some embodiments of the invention, “mismatch-dependent cleavage” can also result in some ancillary cleavage of polynucleotides not involved in a mismatch, so long as cleavage of the mismatched polynucleotides predominates in the reaction. In preferred embodiments of the invention, mismatch-dependent cleavage is substantially specific only for mismatched polynucleotides, with substantially no cleavage of perfectly matched polynucleotides. While in some cases mismatch-dependent cleavage will only recognize certain specific mismatches, in preferred embodiments the reaction will recognize any mismatch involving one or more of the naturally occurring nucleotides.

It is the release of detectable label from polynucleotides involved in mismatched duplexes that distinguishes improved SBH protocols that employ the methods of this invention from previously reported SBH protocols. By releasing the detectable label from mismatched duplexes, a high degree of mismatch discrimination can be achieved. If the method of mismatch cleavage used, e.g., a nuclease or cocktail of nucleases, can recognize any mismatch present, efficient mismatch discrimination can be achieved in manner that is independent of the composition, nature and lengths of the complementary probes. For example, the method attenuates specificity problems caused by the different binding energies of GC and AT pairs. Furthermore, the invention allows the use of less stringent hybridization conditions, because stringency is in effect achieved through mismatch cleavage. The sequencing process is thereby simplified, for instance, because the choice of hybridization and wash conditions becomes less critical to the outcome of the analysis. The use of less stringent conditions also allows the process to be completed more rapidly. The high degree of discrimination against mismatches reduces errors and increases the fidelity of the analysis.

In a preferred embodiment of the present invention, mismatch-dependent cleavage is accomplished by enzymatic cleavage of one or both strands of a mismatched duplex, i.e., enzyme mismatch cleavage (EMC), typically by an endonuclease capable of recognizing a base mismatch. In one embodiment of the invention, a single endonuclease is used. In this embodiment, the endonuclease may recognize one type of base mismatch, e.g., all mismatches involving a particular base, or, preferably, is capable of recognizing more than one type of base mismatch. A number of appropriate endonucleases are known to those of skill in the art and can be obtained from a variety of commercial sources, e.g., Trevigen, Inc. (Gaithersberg, Md. 20877). Endonucleases appropriate for use in the instant invention include, but are not limited to, T4 endonuclease VII, an enzyme capable of recognizing a wide spectrum of base mismatches and cleaving one or both strands involved in the mismatch; E. coli endonuclease V, another enzyme capable of recognizing a variety of base mismatches; MutY enzyme, a DNA glycolase that cleaves the A of an A-G or A-C mismatch; thermostable T-G DNA glycolase (TDG), which recognizes T-G mismatches; and deoxyinosine-3′-endonuclease, which is most effective with A-A and A-C mismatches.

When EMC is achieved with a single endonuclease, it is desirable that the endonuclease be capable of recognizing any potential base mismatch that might occur in the hybridization experiment. Thus, in one embodiment of the invention, a novel, non-naturally occurring endonuclease capable of recognizing a broad range of bases mismatches can be engineered for use in practicing the invention, using recombinant techniques and methodologies well known to one of skill in the art. Such novel endonucleases can be generated, for example, by via site-directed mutagenesis. This method uses oligonucleotide sequences that encode the polynucleotide sequence of the desired amino acid variant, as well as a sufficient adjacent nucleotide on both sides of the changed amino acid to form a stable duplex on either side of the site of being changed. In general, the techniques of site-directed mutagenesis are well known to those of skill in the art and this technique is exemplified by publications such as, Edelman et al., DNA 2:183 (1983). A versatile and efficient method for producing site-specific changes in a polynucleotide sequence was published by Zoller and Smith, Nucleic Acids Res. 10:6487-6500 (1982). Mutagenesis can involve point mutation, insertions, deletions or gene shuffling.

PCR may also be used to create a novel endonuclease. When small amounts of template DNA are used as starting material, primer(s) that differs slightly in sequence from the corresponding region in the template DNA can generate the desired amino acid variant. PCR amplification results in a population of product DNA fragments that differ from the polynucleotide template encoding the polypeptide at the position specified by the primer. The product DNA fragments replace the corresponding region in the plasmid and this gives the desired amino acid variant.

A further technique for generating a novel endonuclease is the cassette mutagenesis technique described in Wells et al., Gene 34:315 (1985); and other mutagenesis techniques well known in the art, such as, for example, techniques described in Sambrook et al. (1989) and Ausubel et al. (1989).

A novel endonuclease with the desired activity may also be produced by screening a pool of endonuclease variants. In one such embodiment of the invention, a library is generated comprising randomly mutagenized nucleic acids encoding an endonuclease and then screened for nucleic acids encoding novel endonucleases possessing the desired base mismatch-specificity, using, for example, the well known phage display methodology, as described in references such as Scott & Smith, Science 249:386-390 (1990) and Devlin et al., Science 249:404-406 (1990).

Novel endonucleases can generated via some form of directed evolution, e.g., gene shuffling and/or recursive sequence recombination, described in U.S. Pat. Nos. 5,605,793 and 5,837,458, incorporated by reference herein in their entirety. For example, using such techniques one can use an endonuclease encoding sequence, or a plurality of endonuclease encoding sequences, as the starting point for the generation of novel sequences encoding functionally and/or structurally similar proteins with altered functional and/or structural characteristics.

The novel endonucleases can be recombinantly expressed and purified by techniques well known to the skilled artisan. See, e.g., Scopes, Protein Purification: Principles and Practice, Springer-Verlag (1994); Sambrook et al. (1989); Ausubel et al. (1989); and U.S. patent application Ser. No. 09/007,300, the disclosures of which are incorporated herein by reference in their entirety.

In a preferred embodiment of the present invention, a cocktail containing a plurality of endonucleases possessing different base mismatch-specificities can be employed. Preferably, the cocktail as a whole will contain endonuclease activities capable of recognizing any base mismatches that might occur in the hybridization experiment, e.g., any mismatch involving the five naturally bases that normally occur in nature (A, C, T, G and U). In a particularly preferred embodiment, the cocktail consists of a variety of different endonucleases, where each individual endonuclease is specific for one particular type of base mismatch and can effectively discriminate a between perfectly matched duplex and a duplex containing a single base mismatch.

Mismatch-specific cleavage can also be accomplished by means of chemical cleavage of a mismatch (CCM). In one embodiment of the instant invention, CCM involves the modification of mismatched cytosines and thymines by treatment with hydroxylamine and osmium tetroxide, followed by cleavage of the modified bases with piperidine. See, e.g., Cotton et al., Proc. Natl. Acad. Sci. (USA) 85:4397-4401 (1998). CCM can also be achieved with less toxic reagants by replacing osmium tetroxide with potassium permanganate in tetraethylammonium chloride, as described by Roberts et al., Nucleic Acids Research 25(16): 3377-78 (1997).

In performing Format II SBH, the cleavage of any labeled target nucleic acid involved in a heteroduplex with an immobilized probe must result in the release of detectable label from the solid substrate. This goal can be accomplished if, after cleavage, the array is washed under conditions where cleaved target nucleic acid is released but uncleaved target remains bound to immobilized probe. Alternatively, release of label from target nucleic acid can be achieved if the target nucleic acid is labeled only at a terminus, and that terminus dissociates from the duplex, regardless of whether the remainder of the target is released from the substrate.

Similarly, in performing Format III SBH, the cleavage of any labeled probe involved in a heteroduplex with a target nucleic acid must result in the release of detectable label from the solid substrate. This end is normally achieved by designing the labeled probe such that label is located only at the free terminus of the composite probe created in the ligation step. In preferred embodiments, therefore, the labeled probe is labeled only at the terminus opposite from the terminus capable of ligation to substrate-bound probe.

In some embodiments of the invention involving Format III SBH, ligase (or other means of ligating contiguous probes) is removed or deactivated prior to cleavage of heteroduplexes by endonuclease. However, removal of ligase prior to addition of endonuclease is not necessary to achieve the desired result. Therefore, in some embodiments of the invention, endonuclease is added in the presence of active ligase, or even concurrently with ligase. In principle, the presence of ligase might interfere with the endonuclease cleavage reaction by reversing the cleavage. However, the presence of mismatched bases near the site of endonuclease cleavage will typically inhibit re-ligation at that site, driving the equilibrium between the two competing reactions towards the formation of cleaved product. Furthermore, by properly adjusting the levels of the competing enzymatic activities, one skilled in the art, without undue experimentation, will be able to provide conditions that result in contiguous probes being ligated and mismatched probes being cleaved.

After cleavage of probes involved in mismatched duplexes, any unattached label is removed by washing. As a consequence of cleaving any probe involved in a mismatched duplex and releasing the label from the substrate, subsequent to the wash step a signal will be only be detectable if the two probes bind contiguously to the target without any mismatches. Determination of the sequence of the ligated probes reveals the sequence of their complementary sequence in the target nucleic acid. The sequences determined from the array of ligated probes can then be assembled to generate the sequence of the entire target nucleic acid, as described infra.

5.6 Detection of Labeled Duplexes After Mismatch-specific Cleavage

Depending on the label or labels used, different methods and apparatus can be employed to detect labeled polynucleotides remaining bound to the solid substrate subsequent to mismatch-specific cleavage. Methods useful for the detection of labeled polynucleotides bound to a solid substrate (commonly referred to as a “gene chip”), have been described and are well known to those of skill in the art.

For radioactive labels, phosphor storage screen technology scanner may be used, for example a Phosphorlmager (Molecular Dynamics, Sunnyvale, Calif.). Chips to be analyzed are put in a cassette and covered by a phosphorous screen. After 1-4 hours of exposure, the screen is scanned and the image file stored in a computer-readable format.

For the detection of fluorescent labels, CCD cameras and epifluorescent or confocal microscopy are used. For the chips generated directly on the pixels of a CCD camera, detection can be performed as described by Eggers et al., Biotechniques, 17(3):516-25 (1994), incorporated herein by reference.

5.7 Determination of the Sequence of the Target Nucleic Acid

Subsequent to the identification of labeled probes that form perfectly complementary matches with corresponding sequences on the target nucleic acid, the data can be assembled to yield the sequence of the target. Analytical methods and algorithms for assembling a set of probe sequences into the corresponding target sequence have been described and are known to those of skill in the art (See, e.g., Dramanac et al., Science 260:1649-52 (1993); Dramanac et al., Electrophoresis 13:566-73; Dramanac et al., J. Biomol. Struct. Dyn. 8:1085-102; Dramanac et al., Genomics 4:114-28 Dramanac et al.)

Data analysis should preferably be performed with the aid of a computer program. In one embodiment involving Format III SBH, the analysis begins from a probe of known sequence, for example a PCR primer that was used to generate the target nucleic acid. Alternatively, when no sequence information is available, one or more positive probes, i.e., bound probes detected after mismatch-specific cleavage, are selected as starting probes. The computer program then identifies positive probes having sequences that contiguously overlap the sequence of the starting probe(s). Preferably, the overlaps are identified in a k-1 fashion (i.e., probes that, when the contiguous region of overlap is aligned, have single nucleotide overhangs); however, in some embodiments (particularly involving accounting for false negative data), k-2 or larger may be used, preferably k-2. The standard k-tuple analysis is described in U.S. Pat. Nos. 5,202,231 and 5,525,464. The process is repeated with the next overlapping sequence, and so on through the entire sequence. Analogous methodology can be used in embodiments involving Format II or other variations of SBH.

Although discussed in terms of de novo sequencing, one of skill in the art will recognize that this method can be used for sequencing even longer targets, if they are similar (preferably >95% similar) to known reference sequences. Specific probes may be used to generate clones or DNA fragment signatures, recognize sequence, score known polymorphisms, and perform others types of DNA sequence analyses.

5.8 Methods of Sequencing By Hybridization in Solution

SBH can also be accomplished by determining the sequence of a probe (or probes) capable of forming a perfectly matched duplex in solution (as opposed to on a solid support) with a target nucleic acid (see, e.g., U.S. patent application Ser. No. 09/227,383). The efficiency and accuracy of such “in-solution” SBH protocols can be improved by using mismatch-dependent polynucleotide cleavage to discriminate between perfectly matched duplexes and mismatched duplexes, particularly single base mismatched duplexes, as described supra.

In performing SBH in solution, the sequences of probes capable of forming perfectly matched duplexes with the target nucleic acid can be determined by means of a variety of techniques known to those of skill in the art. For example, Jurinke et al., Anal. Chem. 69:904-910 (1997), describes the determination of nucleic acid sequences by means of mass spectrometry.

Alternatively, the sequences of probes capable of forming a perfect duplex with a target nucleic acid can be determined by the detection of a label or labels attached to the probe and/or target nucleic acid. In one embodiment of the invention, a duplex can be labeled with two different labels, where decoupling of the two labels as the result of mismatch dependent cleavage indicates that the duplex is mismatched. For example, one nucleic acid strand can be labeled with a fluorescent group, while another strand is labeled with biotin, thereby allowing detection by means of avidin or streptavidin binding. In this example, mismatch cleavage will result in the flourescent group not being associated with streptavidin or avidin binding.

In one embodiment of the invention, analogous to Format III SBH, two labeled probes are covalently attached to one another if (and only if) the probes hybridize to the target nucleic acid at adjacent sites. The probes are labeled in such a manner that a detectable signal is generated if the two probes are attached to one another. This can be accomplished, for example, by labeling each of the probes with one of a pair of fluorophores capable of resonance energy transfer, i.e., the emission spectrum of the first fluorophore overlaps with the excitation spectrum of the second fluorophore. Excitation of the first fluorophore will result in emission from the second fluorophore only if the two probes are covalently attached, thereby indicating that the complementary sequences of the two probes are present at adjacent sites in the target nucleic acid.

In an alternative embodiment, one probe is labeled with a detectable fluorophore, while the other probe contains a group capable of quenching the fluorescent signal of the fluorophore. In this embodiment, the fluorescent signal will be quenched if the two probes are covalently attached, with mismatch-dependent cleavage resulting in the generation of a signal. The fluorescent signal can be used to detect and control for a single base mismatch.

### 6 EXAMPLE

### Use of T4 Endonuclease VII as a Mismatch Cleavage Reagent in Format III SBH

6.1 Materials and Methods

6.1.1 Preparation of Nucleic Acid Reagents

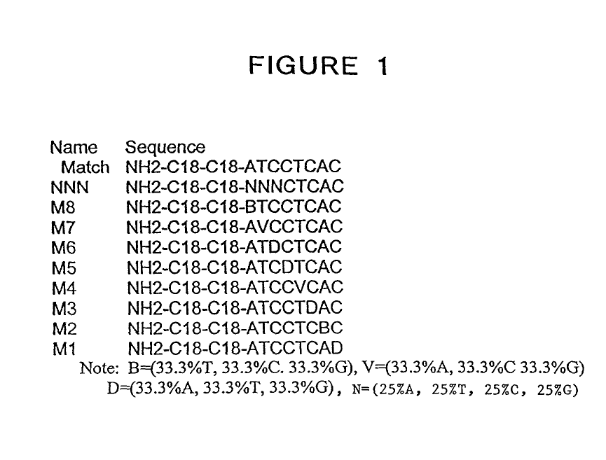

A target nucleic acid (5′-CTTCCAGTGTGATGATGGTGAGGAT-3′; SEQ ID NO: 1), labeling probe (5′-pCATCANN-tamra-3′, where N=25% A, 25% C, 25% G, 25% T), and a series of 8-mer oligonucleotide probes were prepared using conventional methodology. The series of 8-mers included a probe perfectly matched to the target nucleic acid, as well as probes containing single base mismatches at each possible location. See FIG. 1 for a listing of the 8-mer oligonucleotide probes used.

Gene chips were prepared by affixing the series of 8-mer oligonucleotide probes to discrete locations on the surface of a glass slide, thereby forming an array of immobilized probes. Prior to attachment of the probes, the slides were derivatized with 0.2% 1,4-phenylene diisothiocynate (PDC). Solutions of the individual probes (50 pmol/μl of a probe in 0.1 M sodium bicarbonate) were then spotted at discrete locations on the slides. Following spotting, the slides were incubated in a 37° C. humidified oven for 2 hours, washed in 1% sarcosine at 80° C. for 15 minutes on a shaker, rinsed three times with ddH₂O at room temperature, and then spun dry in a centrifuge at 800 RPM for 3 minutes. The slides were then treated with a 1×blocking buffer solution (Amersham #RPN 3601) for 30 minutes at room temperature to prevent nonspecific binding. After blocking, the slides were again rinsed three times with ddH₂O and spun dry.

6.1.2 Hybridization/Ligation Followed by Treatment with T4 Endonuclease VII

Gene chips prepared as described above were hybridized to the target nucleic acid and labeling probe in the presence of T4 DNA ligase. Reaction conditions were as follows: Gene chips were incubated at room temperature in a solution containing 50 mM Tris, 0.05 pmol/μl target nucleic acid, 0.25 pmol/μl labeling probe, 3 units/μl T4 DNA ligase, 0.1 μg/ml bovine serum albumin, 10 mM MgCl₂, 1 mM ATP, pH 7.8 for one hour. Following hybridization/ligation, the gene chips were quickly rinsed with cold buffer and spun dry to remove excess target, labeling probe and ligase. The gene chips were then incubated with 125 units/μl of T4 endonuclease VII in 75 mM PB buffer, 10 mM DTT, pH 6.5 for one hour, after which the gene chips were rinsed in 1% sarcosine, washed with 1% sarcosine and 4×SSPE wash buffer at 80° C. for one hour with shaking, rinsed with ddH₂O and spun dry. Control gene chips were not treated with the endonuclease.

6.1.3 Hybridization/Ligation in the Presence of T4 Endonuclease VII

In these experiments, the gene chips were hybridized to the target nucleic acid and labeling probe described above in the presence of DNA ligase and T4 endonuclease VII. The reaction conditions were as follows: gene chips were incubated at room temperature (for either 1 or 4 hours) in a solution containing 50 mM Tris, 0.05 pmol/μl target. 0.25 pmol/μl labeling probe, 125 units/μl T4 endonuclease VII, 3 units/μl T4 DNA ligase, 0.1 M bovine serum albumin, 10 mM MgCl₂, 1 mM ATP, pH 7.8. The gene chips were then rinsed in 1% sarcosine, washed with 1% sarcosine and 4×SSPE wash buffer at 80° C. for one hour with shaking, rinsed with ddH₂O and spun dry. Control gene chips were treated as above, except that no endonuclease was included in the reaction.

6.1.4 Detection of Labeled Probe Attached to a Gene Chip

The gene chips were scanned in a General Scanner with 90% PMT and 90% laser power. The fluorescence of the TAMRA-label was detected by selecting Cy-3 channel in General Scanner 3000.

6.2 Results

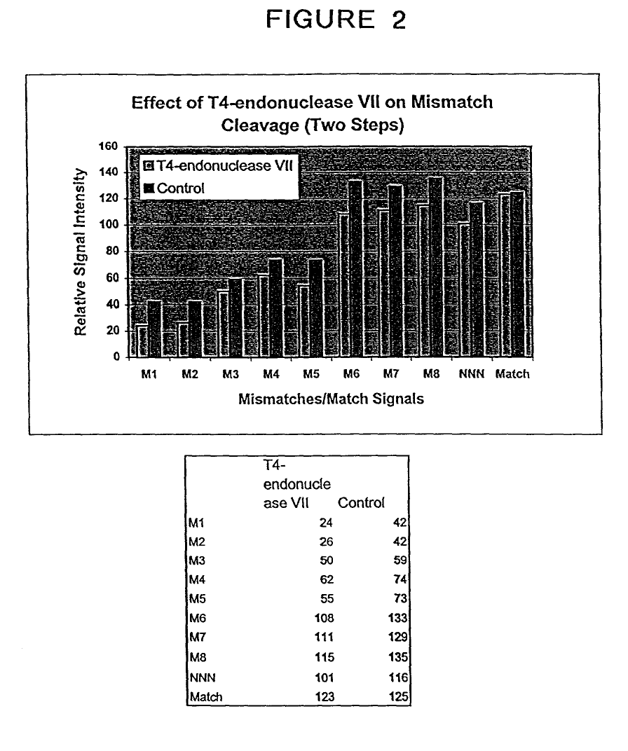

The results of an experiment wherein the gene chip was treated with T4 endonuclease VII following the hybridization/ligation reaction are shown in FIG. 2. It is apparent from the control results that, in the absence of endonuclease, all single base mismatches generate strong signals, especially where the mismatch is located at a position distant from the ligation site, e.g., M6, M7 and M8. Treatment with T4 endonuclease VII has little or no effect on the signal from the perfectly matched probe, but provides varying levels of mismatch discrimination for single-base mismatches at all locations.

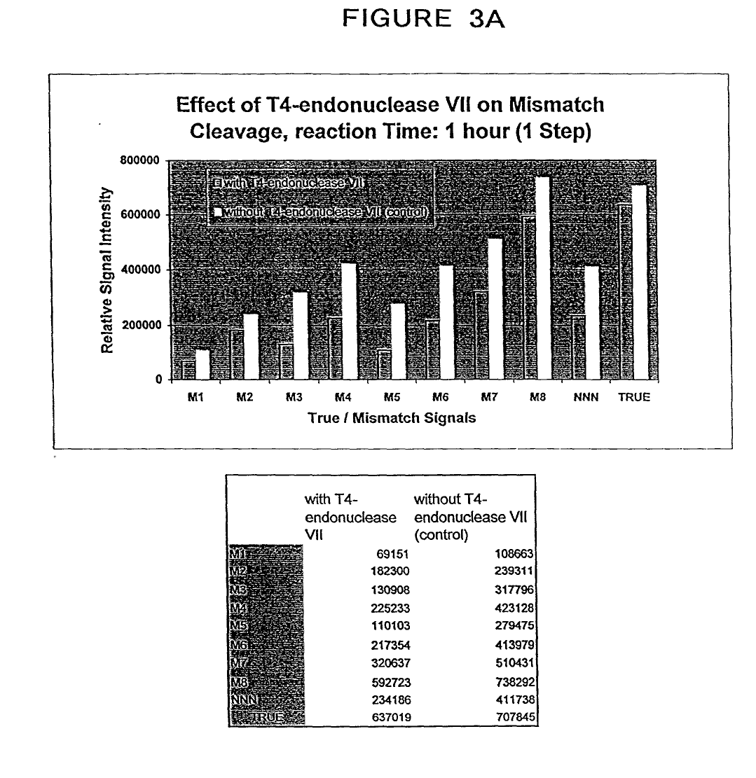

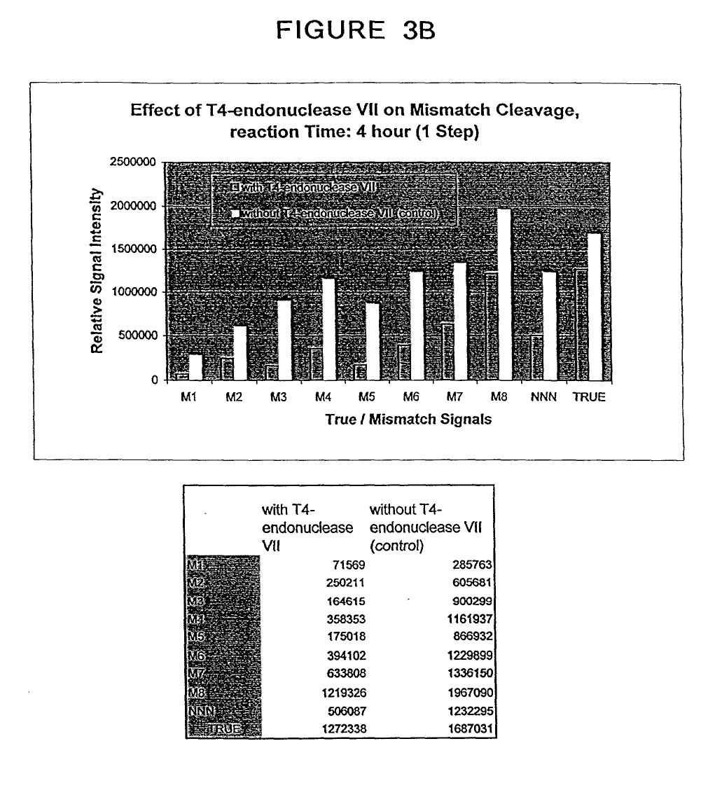

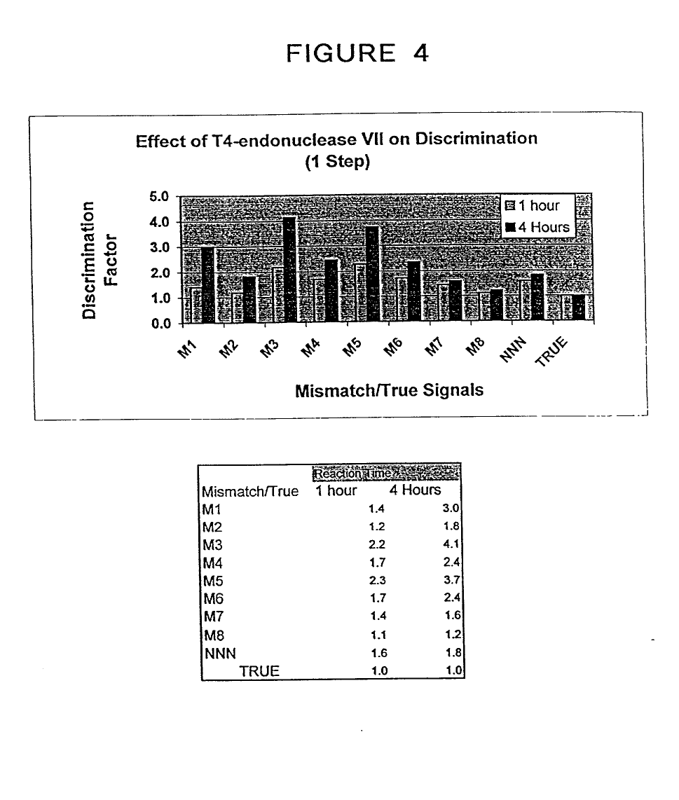

The results of experiments where T4 endonuclease VII was present during hybridization/ligation reaction are shown in FIGS. 3A and 3B. It is evident that treatment with endonuclease during the hybridization/ligation resulted in enhanced mismatch discrimination relative to endonuclease treatment subsequent to hybridization/ligation, and that the four hour reaction resulted in enhanced mismatch discrimination relative to the one hour reaction (see FIG. 4).

The present invention is not to be limited in scope by the exemplified embodiments which are intended as illustrations of single aspects of the invention, and compositions and methods which are functionally equivalent are within the scope of the invention. Indeed, numerous modifications and variations in the practice of the invention are expected to occur to those skilled in the art upon consideration of the present preferred embodiments. Consequently, the only limitations which should be placed upon the scope of the invention are those which appear in the appended claims.

All references cited within the body of the instant specification are hereby incorporated by reference in their entirety.

What is claimed is:

1. A method for identifying a perfectly matched polynucleotide duplex, the method comprising:
(a) providing a polynucleotide duplex comprising two polynucleotides;
(b) treating the polynucleotide duplex under conditions which result in mismatch-dependent polynucleotide cleavage, whereby at least one of the polynucleotides will be cleaved if the polynucleotide duplex is mismatched; and
(c) determining whether at least one of the polynucleotides has been cleaved, wherein a lack of cleavage indicates that the duplex is perfectly matched.

2. The method of claim 1, wherein:
(a) the duplex comprises a first polynucleotide attached to a solid substrate and a second polynucleotide complementary to the first polynucloetide, wherein the first or second polynucleotide is detectably labeled; and
(b) polynucleotide cleavage is determined by determining whether the label is still associated with the solid substrate, wherein continued association of the label with the solid substrate after said treating step indicates that the duplex is perfectly matched.

3. The method of claim 1, wherein the sequence of one of the polynucleotide members of the duplex is known.

4. The method of claim 2, wherein the sequence of one of the polynucleotide members of the duplex is known.

5. The method of claim 1, wherein:
(a) a polynucleotide member of the duplex is labeled with two different detectable labels; and
(b) polynucleotide cleavage is determined by determining whether the two labels have been decoupled, wherein continued coupling of the two labels after said treating step indicates that the duplex is perfectly matched.

6. The method of claim 2, wherein the first polynucleotide is detectably labeled.

7. The method of claim 2, wherein the second polynucleotide is detectably labeled.

8. The method of claim 1, wherein the cleaved polynucleotide is cleaved by a mismatch-specific endonuclease.

9. The method of claim 1, wherein the cleaved polynucleotide is cleaved by a chemical cleavage reagent.

10. The method of claim 8, wherein the mismatch-specific endonuclease is selected from the group consisting of T4 endonuclease VII and CelI endonuclease.

11. The method of claim 8, wherein the mismatch-specific endonuclease is able to recognize a mismatch involving any of the naturally occurring nucleotide bases.

12. The method of claim 1, wherein the cleaved polynucleotide is cleaved with a cocktail comprising a plurality of mismatch-specific nucleases.

13. The method of claim 12, wherein the cocktail is able to recognize a mismatch involving any of the naturally occurring nucleotide bases.

14. The method of claim 2, wherein the detectable label is selected from the group consisting of fluorophores, radioisotopes, enzymes, dyes, EMLs, and ligands capable of detection.

15. The method of claim 14, wherein the detectable label is a fluorophore.

16. The method of claim 14, wherein the fluorophore is TAMRA.

17. The method of claim 2, wherein the detectable label is attached to a terminal nucleotide.

18. A method for releasing a detectable label from a solid substrate, wherein the detectable label is bound to the solid substrate by means of a first polynucleotide, and wherein the detectable label is only released if the first polynucleotide is involved in a mismatched duplex with a second polynucleotide, comprising the steps of:
(a) providing a detectable label bound to a solid substrate by means of a first polynucleotide, wherein the first polynucleotide is involved in a mismatched duplex with a second polynucleotide; and
(b) cleaving the first polynucleotide in a mismatch-dependent manner, whereby the detectable label is released from said solid substrate.

19. The method of claim 18, wherein the first polynucleotide is directly attached to the solid substrate.

20. The method of claim 19, wherein the first polynucleotide is covalently attached to the solid substrate.

21. The method of claim 18, wherein the second polynucleotide is directly attached to the solid substrate, and the first polynucleotide is bound to the solid substrate by means of a complementary base-pairing interaction with the second polynucleotide.

22. The method of claim 21, wherein the second polynucleotide is covalently attached to the solid substrate.

23. A method of determining the sequence of a target nucleic acid, comprising the steps of:
(a) contacting a target nucleic acid with a complementary probe under hybridizing conditions, thereby forming a polynucleotide duplex, wherein the sequence of the complementary probe is known;
(b) treating the polynucleotide duplex under conditions which result in mismatch-dependent polynucleotide cleavage, whereby at least one of the polynucleotides will be cleaved if the polynucleotide duplex is mismatched; and
(c) identifying probes involved in uncleaved polynucleotide duplexes,
wherein the sequence of the target nucleic acid is determined from the sequence of the probes.

24. The method of claim 23, wherein the probe is detectably labeled.

25. The method of claim 24, wherein the polynucleotide duplex is attached to a substrate.

26. The method of claim 24, wherein the polynucleotide duplex is formed in solution.

27. The method of claim 26, wherein the sequence of probes involved in uncleaved polynucleotide duplexes is determined by mass spectrometry.

28. The method of claim 23, further comprising overlapping the sequences determined in step (c) to obtain the full sequence of the target nucleic acid.

29. A method of determining the sequence of a target nucleic acid, comprising the steps of:
(a) contacting a detectably labeled target nucleic acid with a spatially-addressable array of immobilized probes attached to a solid substrate, wherein at least one of the immobilized probes is complementary to the target nucleic acid, and wherein the sequence of the complementary probes is known, thereby forming detectably labeled duplexes between the target nucleic acid and the complementary immobilized probes;
(b) cleaving in a mismatch-dependent manner those target nucleic acids that are involved in a mismatched duplex with an immobilized probe, whereby the detectable label is released from the duplex if and only if the duplex is mismatched; and
(c) identifying probes involved in detectably labeled duplexes,
wherein the sequence of the target nucleic acid is determined from the sequence of the probes.

30. The method of claim 29, further comprising overlapping the sequences identified in step (c) to obtain the full sequence of the target nucleic acid.

31. A method of determining the sequence of a target nucleic acid, comprising the steps of:
(a) contacting a target nucleic acid with
(i) a spatially-addressable array of immobilized probes of known sequence attached to a solid substrate; and
(ii) a detectably labeled oligonucleotide probe of known sequence, under conditions wherein a duplex forms between complementary polynucleotides;
(b) covalently joining adjacently hybridized immobilized probes and labeled probes to form composite probes, thereby attaching the detectable label to the solid substrate;
(c) cleaving in a mismatch-dependent manner those composite probes that are involved in a mismatched duplex with the target nucleic acid, thereby releasing the detectable label from the solid substrate if the composite probe is involved in a mismatched duplex; and
(d) identifying the labeled composite probes remaining attached to the solid substrate after said cleavage,
wherein the sequence of the target nucleic acid is determined from the sequence of the probes.

32. The method of claim 31, comprising overlapping the sequences identified in step (d) to get the full sequence of the target nucleic acid.


SEQUENCE LISTING (1 sequence)

SEQ ID NO: 1 - DNA, 25 bp, Artificial Sequence
  Target nucleic acid
cttccagtgtgatgatggtgaggat